(19) United States
(12) Patent Application Publication
(10) Pub. No.: US 2024/0138694 A1
(43) Pub. Date: May 2, 2024

(54) SENSING BIOPSY NEEDLE

(71) Applicant: Johnson & Johnson Enterprise Innovation Inc., New Brunswick, NJ (US)

(72) Inventors: Joseph Thomas KEYES, Oceanside, CA (US); Matthew KREVER, Basking Ridge, NJ (US); Siobhan CARROLL, Los Gatos, CA (US); Jeffrey D. MESSERLY, Cincinnati, OH (US); James T. SPIVEY, Whitehouse Station, NJ (US)

(21) Appl. No.: 18/495,674

(22) Filed: Oct. 26, 2023

Related U.S. Application Data
(60) Provisional application No. 63/419,530, filed on Oct. 26, 2022.

(51) Int. Cl. A61B 5/053; A61B 5/00

(52) U.S. Cl. CPC A61B 5/053; A61B 5/6848; A61B 5/7264; A61B 2562/043

(57) ABSTRACT

The system and method for characterizing a tissue of interest may include navigating a needle device in proximity to the tissue of interest. The needle device can comprise a member including a plurality of electroconductive segments. The plurality of electroconductive segments can be electrically insulated from one another. The device can also include a first conductive path electrically coupled to a first electroconductive segment. A second conductive path can be coupled to a second electroconductive segment. The method can include contacting the tissue of interest with the first electrode and determining a tissue impedance of the tissue of interest from an electrical signal from the first electrode. The method can also include characterizing the tissue of interest as malignant or benign based on the tissue impedance.

### CROSS REFERENCE TO RELATED APPLICATIONS

This present application claims the benefit of priority under 35 U.S.C. § 119(e) to U.S. Provisional Application No. 63/419,530, filed Oct. 26, 2022, the disclosure of which is hereby incorporated by reference in its entirety for all purposes.

### TECHNICAL FIELD

The present disclosure relates generally to characterizing tissue, and more particularly, to systems and methods for identifying malignant tissue.

### BACKGROUND

Lung cancer is the leading cause of cancer mortality in the world. This is in part because over half of lung cancers are detected in late-stage disease when survival outcomes are very poor. Similar to lung cancer, kidney cancer is among the 10 most common cancers in both men and women. Diagnostic endoscopy is a useful procedure for assessing cancer in both lung tissue and kidney tissue. Generally, diagnostic yield in cancer can vary greatly, which leads to challenges in diagnosing forms of cancer. A number of factors contribute to diagnostic yield, including: lesion size and location, lesion heterogeneity, imaging modalities available to confirm biopsy needle location, and ability to secure adequate tissue with the biopsy needle. Thus, there is a need for intercepting, diagnosing, and treating cancer earlier in the disease progression to improve patient outcomes.

### SUMMARY

Generally, a device for measuring the electrical impedance for a tissue can include a member comprising a plurality of electroconductive segments. The electroconductive segments can be arranged between a proximal end and a distal end of the member. The plurality of electroconductive segments can be electrically insulated from one another. The device can also include a first conductive path. A portion of the first conductive path can be electrically coupled to a first electroconductive segment of the plurality of electroconductive segments, such that the electroconductive segment is configured as an electrode. In some embodiments, the conduit can comprise multiple conductive paths, wherein each conductive paths is electrically coupled to a different electroconductive segment. The device can also comprise at least one insulated segment coupled to at least one of the plurality of electroconductive segments. In some embodiments, the insulated segment can be interspersed between two electroconductive segments of the plurality of electroconductive segments.

The device can comprise a plurality of conduits that can span the length of the member. The plurality of conduits can be equally circumferentially arranged around the member. In some embodiments, the conduit can be helical, such that it wraps around the external surfaces of the electroconductive segments. The helical orientation of the conduit can help reduce mechanical biasing. In other embodiments, at least a portion of the conduit is parallel to a longitudinal axis of the member. Internally, the member comprises a needle that defines a lumen. The device can also comprise a sheath that encapsulates at least a portion of the member. In a further embodiment, the internal surface of the needle is insulated to serve as an electrical conduction barrier when a conductive fixture such as a biopsy extraction fixture traverses the lumen.

Generally, a system for measuring the electrical impedance for a tissue can include a processor and a member comprising a plurality of electroconductive segments. The electroconductive segments can be arranged along a length dimension of the member. The plurality of electroconductive segments can be electrically insulated from one another. The device can include a conduit defined by an exterior surface of the member and extends along at least a longitudinal portion of the member. The device can also include a first wire oriented in the conduit and electrically coupled to a first electroconductive segment. A second wire can be oriented in the conduit and electrically coupled to a second electroconductive segment. The first electrode and the second electrode are each configured to provide an electrical signal, and the processor is configured to determine a tissue impedance value based on the electrical signals associated with the first electrode and the second electrode. In other embodiments, the system can comprise a suction device or extraction fixtures configured to remove a tissue through the lumen.

A method for characterizing a tissue of interest may include navigating a needle device in proximity to the tissue of interest. The needle device can comprise a member including a plurality of electroconductive segments. The electroconductive segments can be arranged between a proximal end and a distal end of the member. The plurality of electroconductive segments can be electrically insulated from one another. The device can also include a first conductive path oriented in the conduit and electrically coupled to a first electroconductive segment. A second conductive path can be oriented in the conduit and electrically coupled to a second electroconductive segment. The method can include contacting the tissue of interest with the first electrode and determining a tissue impedance of the tissue of interest from an electrical signal from the first electrode. The method can also include characterizing the tissue of interest based on the tissue impedance.

In other embodiments, characterizing the tissue of interest can include determining the tissue of interest to be malignant or benign. Further, determining the tissue impedance comprises identifying an electrical impedance value associated with the tissue of interest and transmitting the electrical signal associated with the electrical impedance value to a computing device. In other embodiments, the method can comprise delivering a solution to the tissue of interest via the needle device. The method can also include extracting a sample of the tissue of interest via the needle device. The method can comprise repositioning the needle device relative to the tissue of interest. In other embodiments, repositioning the needle device can comprise changing an insertion depth of the needle device in the tissue of interest, and changing an insertion angle of the needle device in the tissue of interest. Repositioning the needle device can also be based on characterizing at least a portion of the tissue of interest as benign tissue or as malignant tissue.

The method can comprise locating a boundary of malignant tissue between the first and second tissue regions based on the first tissue impedance and the second tissue impedance. In other embodiments, the method further comprises determining a length dimension of malignant tissue based on the first tissue impedance and the second tissue impedance. The method can comprise placing the needle device in a bronchoscope and positioning the bronchoscope in proximity to the tissue of interest. The method can also comprise determining additional electrical measurements at each electrode.

### BRIEF DESCRIPTION OF DRAWINGS

FIG. 1 depicts an exemplary system for measuring tissue impedance.

FIG. 2 depicts an isometric view of an example embodiment of a needle device.

FIG. 3 depicts a partial isometric view of an example embodiment of the needle device.

FIG. 4 depicts a front view of an example embodiment of a needle device.

FIG. 5 depicts an exploded view of two segments in an example embodiment of a needle device.

FIGS. 6A-6C depict alternative side cross-sectional views of a plurality of segments coupled together in an example embodiment of a needle device.

FIG. 7 a side cross-sectional view of a plurality of segments coupled together in an example embodiment of a needle device.

FIG. 8 depicts a detailed view of a conduit with wires at a first segment of an example embodiment of a needle device.

FIG. 9 depicts a detailed view of the conduit with wires at a second segment of the needle device depicted in FIG. 8.

FIG. 10 depicts a detailed view of the conduit with a wire at a third segment of the needle device depicted in FIG. 8.

FIG. 11 is a flow diagram of a method for characterizing a tissue of interest.

FIG. 12 is an illustrative schematic of a method for characterizing a tissue of interest.

FIG. 13 is a graph depicting a relationship between Cole relaxation frequency and likelihood of malignant tissue.

FIG. 14 is an illustrative schematic of a method for extracting a sample from the tissue of interest.

### DETAILED DESCRIPTION

It is to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated to some other value approximately or about the same. It is generally understood, as used herein, that it is the nominal value indicated ±10% embodiment unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the disclosure as well as the devices themselves to be used within the methods disclosed herein. These and other materials are disclosed herein, and it is understood that when combinations, subsets, interactions, groups, etc. of these materials are disclosed, that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible, unless specifically indicated to the contrary. Thus, if a class of materials A, B, and C are disclosed as well as a class of materials D, E, and F and an example of a combination material, A-D is disclosed, then even if each is not individually recited, each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the disclosure. Thus, if there are a variety of additional steps that can be performed, it is understood that each of these additional steps can be performed with any specific aspect or combination of aspects of the methods of the disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

### System

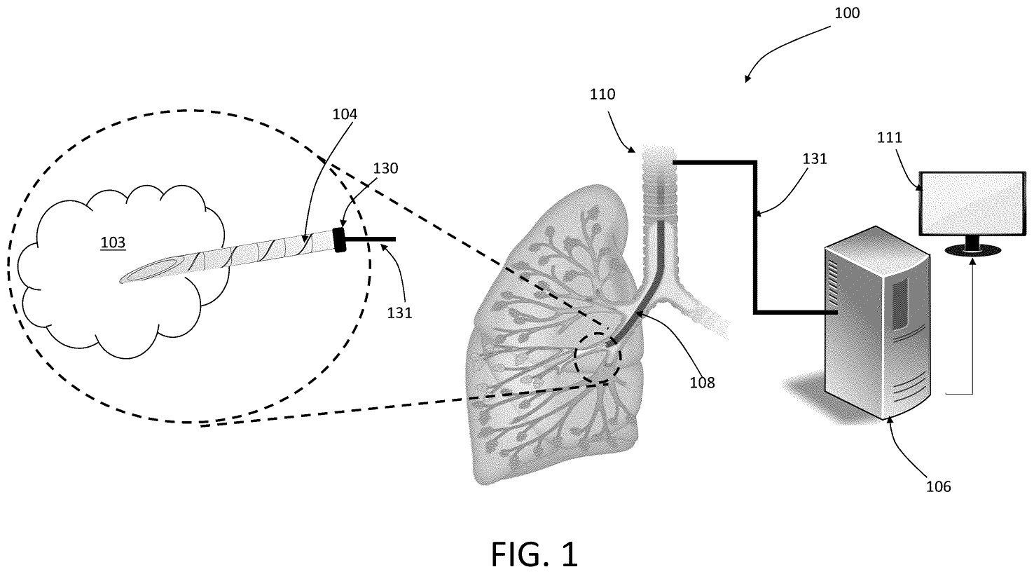

The system 100, as depicted in FIG. 1, can comprise multiple components to perform analysis on and/or interact with the tissue of interest 103. The system 100 comprises a needle device 104 that can function as a multi-purpose tool. For example, the needle device 104 can (i) measure one or more electrical properties of the tissue of interest using one or more sensing regions, such as for characterizing the tissue of interest as likely healthy, malignant, or benign, (ii) extract a substance (e.g., a biopsy sample or fluid) from the tissue of interest (e.g., via aspiration), and/or (iii) deliver a substance (e.g., therapeutics) to the tissue of interest. During the measurement of electrical properties, the needle device 104 allows for physical and electrical contact between the needle device's exterior surface and the tissue of interest 103 to acquire a high integrity electrical signal (with reduced noise) from the tissue. The configuration of system 100 enables measurement of electrical properties across a larger region of tissue, rather than at a single location. In an exemplary embodiment, the needle device 104 can comprise multiple electrodes that permit the system 100 to gather information that assists identifying malignant cells within the tissue of interest.

The tissue of interest can be identified, for example, in a prebiopsy assessment by an imaging technology such as computed tomography, ultrasound, and/or the like. Once identified, the tissue of interest 103 can indicate a region of the body that requires further inspection and/or treatment. This further inspection can comprise a characterization of the bioelectric properties of the tissue (e.g., lung or kidney tissue). Characterizing the bioelectric properties can be based on modeling the tissue as an equivalent electrical circuit. The system 100 can be used to measure the bioelectric properties of the tissue by defining the properties relative to the equivalent electrical circuit. For example, the bioelectric property may be tissue impedance, wherein the impedance is related to the restriction of electricity flow through the circuit (e.g., through lung or kidney tissue). The system 100 can comprise a needle device 104 and a computing device 106. The needle device 104 can engage the tissue of interest 103 to take impedance measurements and communicate those measurements to the computing device 106 as electrical signals (e.g., voltage, current, etc.). The computing device 106 can receive the electrical signal(s) from the needle device 104 and analyze the electrical signals to assess whether the tissue of interest is healthy, malignant, or benign.

The system 100 assessment of the tissue of interest 103 can be based on the electrical impedance generated by the tissue of interest, as described in further detail herein. In particular, as further described herein, malignant cells typically yield a different impedance value than cells that are benign or healthy. In a further aspect, the system can be configured for in vivo use or in vitro use. During in vivo use, the system 100 can further comprise or be used with an endoscope 108, which may, for example, be controlled manually and/or with robotic assistance to help place the needle device 104 in or near the tissue of interest 103. For use that involves tissue of interest in the lung cavity, the endoscope 108 can be a bronchoscope. For example, as shown in FIG. 1, a bronchoscope can be used to pass the needle device 104 into the trachea 110 of the patient. However, for other applications, the endoscope and/or needle device 104 can be sized and/or shaped for navigation in any suitable tissue environment. For example, the endoscope may be a ureteroscope for aiding placement of the needle device 104 in or near tissue of interest in a kidney.

### Needle Device

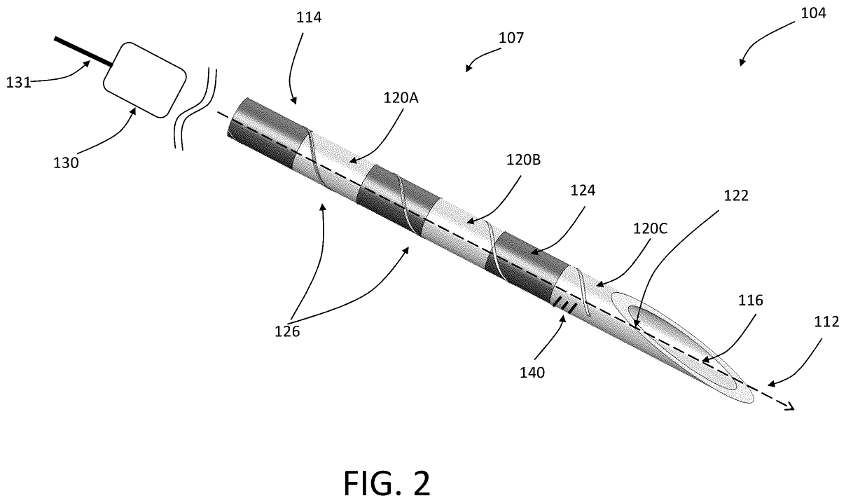

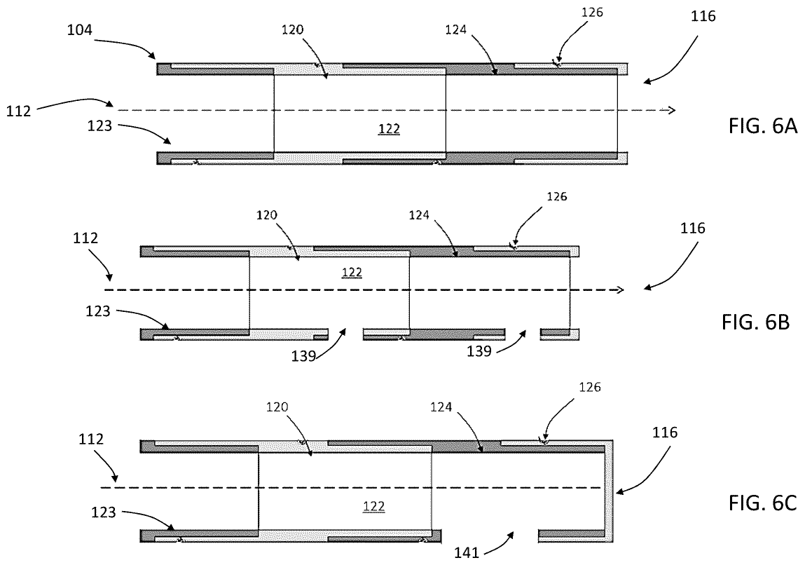

Referring to FIG. 2, the needle device 104 can include a member 107 with a proximal end 114 and distal end 116. In one aspect, the member 107 can comprise a length dimension oriented along a longitudinal axis 112. The longitudinal axis 112 can be oriented along the span between the proximal end 114 and the distal end 116. In one aspect, the member 107 can be primarily linear in construction along the longitudinal axis. In other embodiments, the member 107 of the needle device 104 can comprise one or more curved regions. Further, as shown in FIG. 2, the member 107 of the needle device 104 can comprise a plurality of electroconductive segments 120 (e.g., segments 120A-C). As depicted in FIG. 2, the electroconductive segments 120 can be arranged in series along the longitudinal axis of the member 107. The internal surface 123 of the segments can also define a lumen 122, as shown in FIG. 4. The lumen 122 can extend along through the member 107 along the longitudinal axis 112 and can terminate at a distal end 116 of the needle device 104. Additionally or alternatively, as depicted in FIG. 6B, internal surface 123 can define at least one capillary lumen 139 that branches away from the primary lumen 122 at an angle relative to the longitudinal axis 112. Further, the internal surface 123 can define an opening on a side of the external surface of the needle device 104, providing access to the capillary lumen 139. In yet another embodiment, primary lumen 122 can provide a pathway for substances and/or extraction fixtures to engage the tissue of interest. In a further embodiment, as depicted in FIG. 6C, the needle device 104 can also define an opening for the primary lumen 122 that exits the needle device 104 at a side surface 141 instead of at a distal end 116 of the needle device 104. In some embodiments, the distal end 116 of the needle device 104 can have a sharpened (e.g., beveled) tip, such as to help pierce tissue and facilitate access to and/or within the tissue of interest.

Functionally, the segments 120 can be electroconductive such that an individual segment may function as an electrode. When the electrode defined by the segment comes in contact with the tissue of interest, an impedance value of the tissue can be determined based on an electrical signal from the electrode. In some embodiments, the member 107 may be comprised of multiple electroconductive segments 120, and an impedance value can be determined for each respective electroconductive segment. As depicted in FIG. 2, the segments 120A-C can have a uniform length. In other embodiments, the lengths of the segments 120A-C along the longitudinal axis 112 can be variable with respect to each other. The segments 120A-C can also comprise various materials, wherein each segment can comprise a different material. For example, the segments 120A-C can comprise metallic (e.g., medical grade stainless steel), ceramic and/or composite material(s), wherein at least a portion of the segment 120A-C is electroconductive material. Suitable materials for metallic material include any suitable biocompatible metal such as Pt, Pt-Ir, nitinol, stainless steel, etc. Furthermore, in some embodiments, one or more of the segments 120 can have a roughened surface for improved electrical contact.

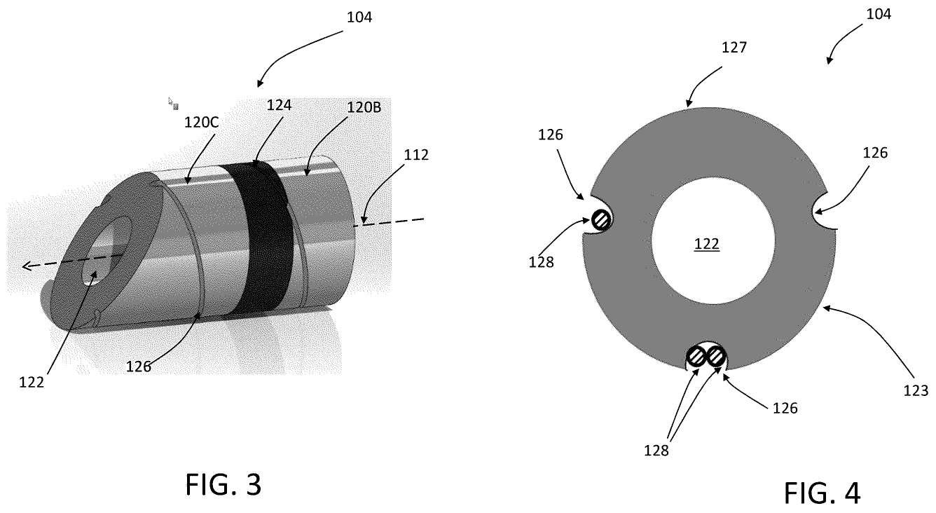

Further, each segment 120 can also be electrically insulated from the adjacent segment. Electrically insulating adjacent segments from one another can ensure that the adjacent segments are distinguishable with respect to the electrical measurements they can receive. In some embodiments, the member 107 can comprise one or more insulated segments 124 that are interspersed between two electroconductive segments 120. As depicted in FIG. 3, an insulated segment 124 can separate two electroconductive segments 120B, 120C. The insulated segment 124 placed between two electroconductive segments can maintain an electrical boundary between electrical circuits defined by the two electroconductive segments, such that the electroconductive segments can provide distinct electrical signals (e.g., enable distinct impedance measurements). The insulated segment 124 can comprise a biocompatible material that is insulative/restricts the flow of electricity, such as rubber, silicone, plastics, or the like (e.g., PEEK, polycarbonate, nylon, etc.). Additionally or alternatively, two or more electroconductive segments 120 can be electrically insulated from each other via an insulative coating (e.g., parylene or PTFE) at their mating or abutting interfaces.

In some embodiments, one or more of the electroconductive segments 120 and/or insulated segments 124 can include a radiopaque material that can, for example, aid visualization of the needle device 104 using fluoroscopic X-ray imaging techniques (e.g., to visualize needle location, shape, and/or orientation within a patient). For example, one or more of the electroconductive segments 120 may include platinum, which is radiopaque. Additionally or alternatively, at least a portion of the needle device 104 may have one or more echogenic features such as surface roughness, for improved visibility using ultrasound imaging techniques. For example, one or more of the electroconductive segments 120 and/or one or more of the insulated segments 124 may include a suitable roughened surface.

As depicted in FIG. 2, in some embodiments, the needle device 104 (or a structure coupled to the needle device) can include one or more reference features 140 that indicate position of the needle device relative to a reference position. For example, the one or more reference features 140 can indicate the distance between the distal end of the needle device relative to the distal exit end of an endoscope 108 working channel through which the needle device 104 is inserted. In some embodiments, such a reference feature may be located at a distal end of the needle device 104 that can be visible after the distal end of the needle device exits the endoscope working channel (e.g., a radiopaque reference feature visible using fluoroscopic X-ray imaging techniques). Additionally or alternatively, a reference feature 140 may be located at a proximal end 114 of the needle device (or structure coupled to a proximal portion of the needle device) and visible outside the patient. The one or more reference features 140 may have any suitable shape (e.g., ring or band shaped, arcuate, dash, notch, etc.) at particular axial location(s). Additionally or alternatively, various reference features may include text (e.g., numbers or letters), symbolic patterns (e.g., solid line, dashed line), suitable dimensions (e.g., width) and/or the like, to help an operator distinguish between different reference features. The one or more reference features can be formed via laser etching, printing, and/or in any suitable manner.

In an embodiment, the needle device 104 can yield additional measurement configurations when multiple electrodes are oriented along the longitudinal axis. In taking certain electrical property measurements, the electroconductive segments 120A-C can selectively function as either a lead electrode or a reference electrode. For example, the first electroconductive segment 120A can be the reference electrode and the third electroconductive segment 120C can be the lead electrode, facilitating an impedance measurement across the two electrodes. In other embodiments, the roles of the lead electrode and reference electrode can be switched between the first electroconductive segment 120A and third electroconductive segment 120C. Similarly, combinations including the second electroconductive segment 120B with the first electroconductive segment 120A or third electroconductive segment 120C can be used to define the reference and lead electrode. In further embodiments, the needle device can comprise fewer than three electroconductive segments (e.g., two), or more than three electroconductive segments (e.g., four, five, six, seven, eight, nine, ten, or more). In some of such embodiments, the role of the lead or reference electrode can be switched between segments as described above. Switching the lead and reference electrodes along the length of the member 107 can also facilitate additional measurements based on the depth of insertion in the tissue of interest.

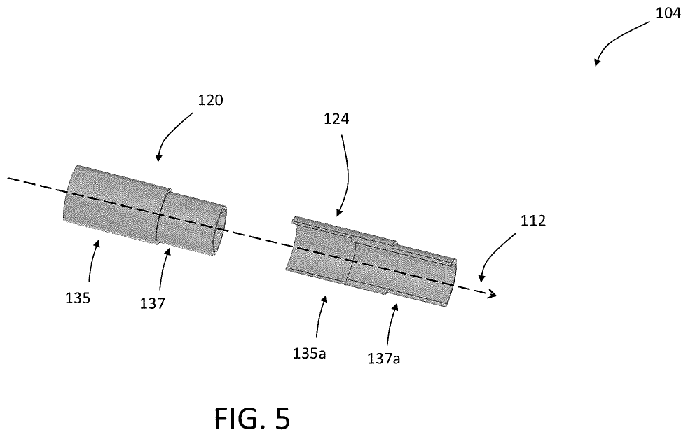

In embodiments in which the member includes one or more insulating segments 124 interspersed between electroconductive segments 120, the electroconductive and insulating segments can be coupled to each other in multiple ways. In some embodiments, the electroconductive and insulating segments can have corresponding mating interfaces and/or engage each other with a suitable mechanical interfit. For example, in one aspect, an end region of a first segment can have a smaller cross section than an end region of a second segment adjacent the first segment, such that at least a portion of the first segment can be nested within the second segment as shown in FIG. 5 (e.g., the first segment may include an insert region that is received within a receptacle region of the second segment). Although FIG. 5 depicts segments having stepped changes in diameter, it should be understood that in some embodiments, the mechanical interfit may include engagement between surfaces of any suitable shape (e.g., tapered, etc.). In some embodiments, the electroconductive and insulating segments of the needle device 104 can be separately formed (e.g., molded, machined, etc.). The electroconductive and insulated segments can then be assembled together in an axially aligned, stacked manner, as depicted in FIG. 6A. The segments may additionally or alternatively be coupled to each other through swaging, epoxy, and/or other suitable techniques. Instead of separately formed segments that are coupled to one another in an assembly, the alternating metal and insulating segments may alternatively be formed through a suitable overmolding process.

In some embodiments, the segments can comprise at least one conduit 126 (or other suitable channel) defined in the exterior surface of the member, such as in the exterior surface of one or more electroconductive and/or insulating segments. As depicted in FIG. 4, the conduit 126 may function to carry at least one wire 128 (and/or other suitable conductive path(s)) to and/or from one or more electroconductive segments. As depicted in FIG. 2, one embodiment of the conduit 126 can be a conduit that extends along at least a longitudinal portion of the member 107. In a further aspect, the conduit 126 can comprise a helical or spiral pattern such that the portion of the conduit revolves around the member 107 of the needle device along the longitudinal axis. The helical pattern can be used to reduce the mechanical bias. In embodiments in which the conduit 126 is oriented in the helical pattern, the pitch of the conduit 126 may depend on factors such as the size and/or spacing of the metal segments. For example, in some embodiments, the conduit 126 can be longitudinal, such as oriented to be parallel to the longitudinal axis of the member 107.

The conduit 126 may be formed in the surface of the member in various manners. In some embodiments, at least a portion of the conduit 126 may be formed after some or all of the electroconductive and/or insulating segments are assembled. For example, a conduit 126 can be mechanically ground or etched (e.g., via laser or chemical etching processes) into the outer surface of the assembled needle device 104. Additionally or alternatively, in other embodiments, a portion of the conduit 126 can be formed into the surface of one or more electroconductive and/or insulating segments prior to assembly (e.g., via mechanical grinding, laser etch, chemical etch, etc.), and such electroconductive and/or insulating segments may be aligned so as to align the portions of the conduit 126 and enable the conduit 126 to be continuous along the member.

In a further aspect, the member 107 can define multiple conduits 126 in the external surface of the segments. The multiple conduits can be spaced equidistant relative to each other around the longitudinal axis (e.g., around the perimeter or circumference of the member of the needle device). For example, as depicted in FIG. 4, a member has a circular cross-section and has three (3) conduits, and each conduit is equidistant (e.g., the central angle with respect to each conduit is 120 degrees) from each other around the perimeter or circumference of the needle. The radially equal distribution of multiple conduits 126 may, for example, help reduce mechanical biasing in the member that may otherwise occur. However, in other embodiments, the multiple conduits are not equidistant from another. In a further aspect, each conduit 126 can house one or more conductive paths 128 configured to transmit electrical signals and electrical current.

In some embodiments, the conduit 126 can have any suitable cross-sectional shape. As depicted in FIG. 4, the conduit 126 can comprise a semi-circular cross-sectional shape defined in the exterior surface 127 of member 107. In other embodiments, the cross-section of the conduit 126 can comprise other geometric shapes, including but not limited to triangle, square, rectangle, rhombus, and the like. The diameter and/or depth of the conduit 126 may depend on, for example, the number and/or size of the lead wires required for the number of electroconductive segments in the needle device. In some embodiments, for example, the width or depth of the conduit can range from less than about 0.01 mm to about 2 mm.

Referring to FIGS. 1, 2, and 4, the needle device 104 can further comprise one or more conductive paths 128 in electrical communication with the one or more electroconductive segments. A conductive path is a physical component or feature that can carry electrical signals from an electroconductive segment and away from the distal end of the member (e.g., to an end connector 130). The end connector can comprise electrical connectors that are configured to electrically couple the conductive path to the computing device 106. For example, connector wires 131 originating from computing device 106 can be physically coupled to the end connector 130, completing an electrical connection between the conductive path 128 and computing device 106. In a further aspect, the end connector 130 can be configured to distinguish the distinct electrical signals received from each electrode and transmit the distinct signals to the computing device via the connector wires 131.

In some embodiments, the needle device 104 may include a plurality of conductive paths including a first conductive path in electrical communication with a first electroconductive segment, a second conductive path in electrical communication with a second electroconductive segment, etc. In some embodiments, an end connector 130 is located, for example, at the proximal end 114 of the needle device 104 or extends to the computing device 106 that processes an electrical signal. The conductive path can serve as the electrical pathway to carry the signal from the electrode to the end connector 130. For example, the conductive path can carry the electrical signal that may be analyzed to indicate the electrical impedance of tissue in contact with an electroconductive segment associated with that conductive path. The conductive path can be a wire 128, wherein the wire 128 is at least partially insulated. In some embodiments, the wire 128 can terminate at the end connector 130. Where the wire 128 terminates, the end connector 130 may comprise electrical couplings that are configured to be connected to the computing device 106 to analyze the signal carried by the wire. In other embodiments, the conductive path can also comprise an electrical path integrated into the surface of the needle device. For example, the conductive path 128 can be formed on the surface of the needle device (e.g., electrical trace) through etching, plating, printing, and/or other suitable semiconductor process(es). In a further aspect, the conductive path 128 formed (e.g., being etched, plated, or printed) or engaged on the exterior surface (e.g., coupled or adhered) of the member 107 may be structured without a conduit 126 or channel defined by the exterior surface 107 of the member.

In embodiments in which the conductive path is a wire, the wire can be oriented to rest within the conduit 126 defined by the external surface of the segments 120. As shown in FIG. 4, in some embodiments, a single wire 128 may be positioned in a single conduit 126, and/or multiple wires 128 may be positioned in a single conduit 126. Furthermore, in embodiments in which the needle device 104 comprises multiple conduits 126, a single wire 128 or multiple wires can also be placed in each respective conduit. One or more of the wires 128 can be insulated such that the wires are not shorted to each other or result in interference between the respective electrical signals carried by each wire. In some embodiments, the wire 128 can have a generally rounded profile (e.g., circular, elliptical) or flat profile (e.g., square, rectangular), or a combination of rounded and flat profiles (e.g., semi-circular, semi-elliptical).

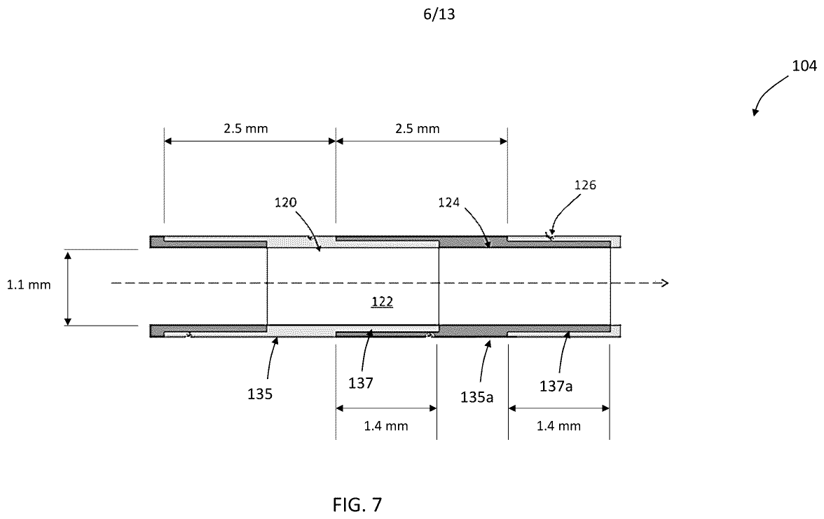

In some embodiments, the size and/or shape of the needle device 104 and its features may be configured based at least in part on characteristics of the tissue environment to be used. For example, FIG. 7 depicts an example embodiment of a needle device 104 that may be sized and/or shaped for accessing tissue of interest in and/or around lung tissue. As shown in FIG. 7, the interior surfaces of the needle device 104 can define the lumen 122, wherein the lumen diameter can be about 1.1 mm. In some embodiments, the lumen diameter can range from between about 0.5 mm to about 1.5 mm. In the exemplary embodiment as shown, the electroconductive segments 120 and insulated segments 124 are coupled in a stacked configuration. Each segment can have a segment length of between about 1 mm to about 3 mm. Further, as depicted in FIG. 5, a segment 120 can be sub-divided into two regions, a receptacle region 135 and insert region 137. Each of the receptacle region 135 and the insert region 137 can have any suitable length, though in an example embodiment the length of the exterior surface of the receptacle region 135 can be about 2.5 mm. In some embodiments, the exterior receptacle region length can be any suitable portion of the overall segment length (e.g., 20%, 25%, 30%, 50%, 75%, etc.). The length of the insert region 137 can be about 1.4 mm, for example, though in general the length of the outer surface of the insert region can include the remaining length of the overall segment 120 that is not part of the receptacle region. In the stacked configuration of the needle, the insert region of a first segment will nest inside the receptacle region of a second segment (e.g., the inner surface dimension [perimeter/circumference] of the receptacle section will be greater than the outer surface dimension [perimeter/circumference] of the insert section).

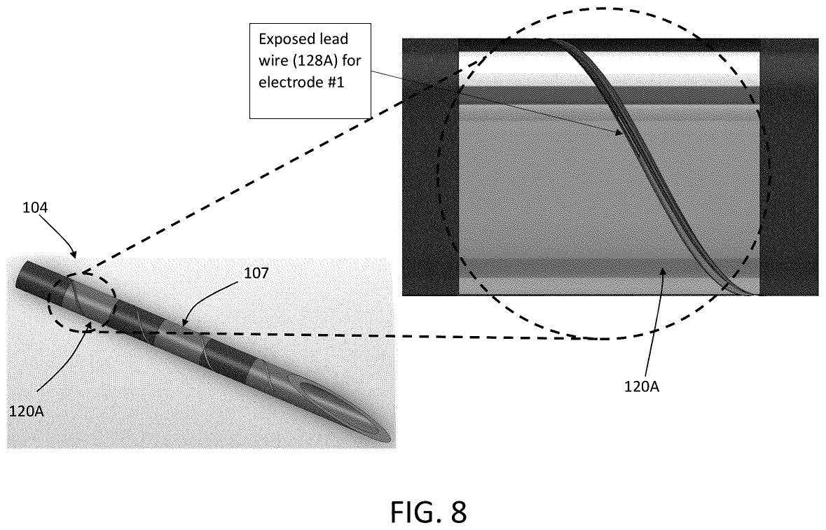

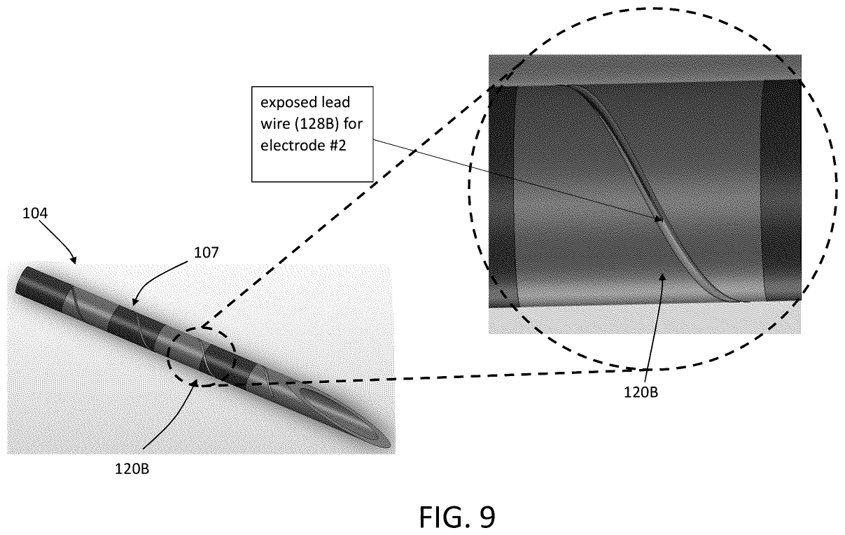

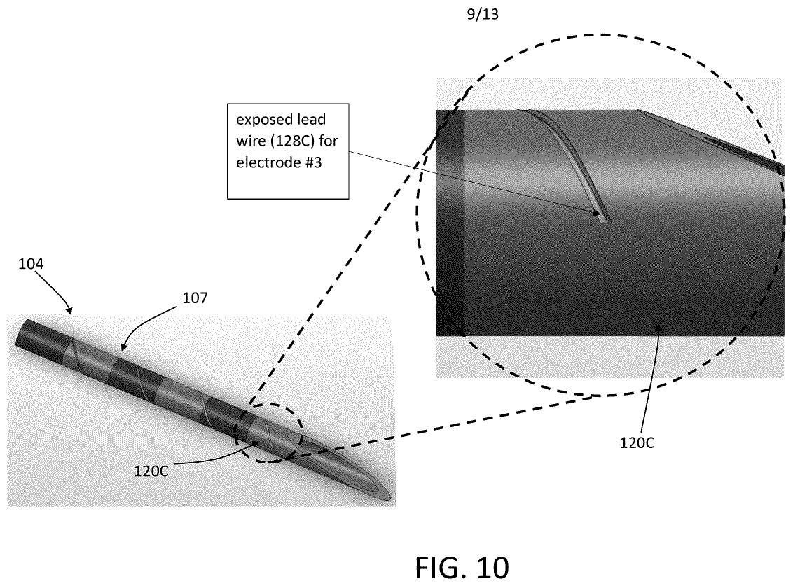

In some embodiments, a portion of each wire 128 may be in electrical contact with a respective electroconductive segment on the member, such that the wire 128 may function to carry electrical signals from its respective electroconductive segment. Where each wire 128 reaches its respective segment, as depicted in FIGS. 8-10, a portion of the wire can be electrically exposed such that there is no insulation on the wire in that location. For example, in some embodiments, the distal end of a wire 128 may be stripped and exposed to enable electrical contact between the stripped distal end of the wire 128 and a respective electroconductive segment. In some embodiments, at least a portion of the wires 128 may have different lengths (e.g., the wires 128 may terminate at their distal ends where they are electrically coupled to their respective electroconductive segments at different longitudinal locations along the member).

As another example, in some embodiments, a wire 128 may be stripped and exposed at an intermediate location along its length (e.g., between its proximal and distal ends), to enable electrical contact between the stripped intermediate portion of the wire 128 and a respective electroconductive segment. In other words, instead of the wire terminating at the electroconductive segment, only a portion of the wire is electrically exposed and coupled to the electroconductive segment and the remainder of the wire may continue distally along the member. In some of these embodiments, the wires 128 may generally have the same length, which may, for example, help improve radial structural symmetry for further reduced mechanical biasing. The remainder of all wires after the exposed intermediate wire potions may extend, for example, to the distal end of the conduit or distalmost electroconductive segment. Alternatively, in some embodiments in which at least a portion of multiple wires 128 have exposed intermediate wire portions, at least a portion of such wires 128 may have different overall lengths.

In some embodiments, the exposed portion (e.g., end or intermediate portion) of a wire 128 can be electrically coupled to the surface of the electroconductive segment 120 with a suitable attachment technique such as laser welding, soldering, and/or the like, thereby allowing the electroconductive segment to be configurable as an electrode. In some embodiments, the conduit may additionally or alternatively be filled with a potting compound (e.g., epoxy potting compound) to help secure the wire(s) 128 within the conduit.

The needle device can also comprise one or more extraction fixtures. The extraction fixtures are components that can pass through the lumen of the member at the distal end of the member and engage the tissue of interest. In a further aspect, these extraction fixtures can comprise hooks, tines, cryoprobe, and/or other suitable tools that can be used to remove a portion of the tissue. In another embodiment, the inner surface 123 that defines a lumen 122 can be electrically insulated to prevent any unintended changes in electrical measurements when these extraction fixtures are used, in particular if the extraction fixtures are electrically conductive.

A protective layer, e.g., an insulating polymer tube (sheath) can also be provided over the needle device 104 to protect the needle from environmental factors such as mucus, etc., until the needle is ready to be used for sensing, biopsy, etc.

In some embodiments, the needle device can be coupled to an external pump or another suitable device for facilitating the removal and/or delivery of substances to a region of interest of a patient after the needle device is placed in the patient (e.g., proximate to tissue of interest). For example, the needle device 104 can be coupled to a suction device (e.g., external to the patient) that may be actuated to aspirate tissue and/or fluid(s) from tissue through one or more lumens of the needle device. As another example, the needle device can be coupled to a pressure pump that may be actuated to deliver fluid or other suitable substances (e.g., therapeutics for treating the tissue of interest, electrolyte solution that may aid in enhancing electroconductivity of the tissue of interest).

In some embodiments, a cryoprobe (e.g., 1 mm cryoprobe) may be introduced through the lumen 122 of the needle device 104. The cryoprobe may be used to anchor the needle device 104 to the tissue of interest 103 to minimize or eliminate needle device movement (thus stabilizing the needle device) as the electrical signal is acquired, and/or a biopsy sample is taken. Other methods for anchoring or stabilizing a needle device to the tissue includes mechanical anchor, chemical glue, or other suitable methods. The cryoprobe can withdraw through the needle lumen (e.g., lumen 122). In some embodiments, the needle device 104 may also comprise a separate suction device to aid in the extraction of tissue or fluids. Such a suction device may be passed through the needle lumen.

### Computing Device

The computing device 106 can include one or more processor(s) (e.g., central processing units (CPUs), graphical processing units (GPUs), holographic processing units (HPUs), etc.) The processors can be a single processing unit or multiple processing units in a device or distributed across multiple devices (e.g., distributed across two or more computing devices). The computing device 106 can include one or more input devices that provide input to the processors, notifying them of actions. The actions can be mediated by a hardware controller that interprets the signals received from the input device (e.g., the needle device 104) and communicates the information to the processors using a communication protocol. The processors can be coupled to other hardware devices, for example, with the use of an internal or external bus, such as a PCI bus, SCSI bus, wireless connection, and/or the like. The processors can communicate with a hardware controller for devices, such as for a display 111. The display 111 can be used to display text and graphics (e.g., impedance measurement results). In some implementations, the display 111 includes the input device as part of the display, such as a touchscreen. Other I/O devices can also be coupled to the processor, such as a network chip or card, video chip or card, audio chip or card, USB, firewire or other external device, camera, printer, speakers, CD-ROM drive, DVD drive, disk drive, etc.

The computing device 106 can include a communication device capable of communicating wireles sly or wire-based with other local computing devices or a network node. The communication device can communicate with another device or a server through a network using, for example, TCP/IP protocols. The computing device can utilize the communication device to distribute operations across multiple network devices. The processors can have access to a memory, which can be contained on one of the computing devices. A memory includes one or more hardware devices for volatile or non-volatile storage, and can include both read-only and writable memory. For example, a memory can include one or more of random-access memory (RAM), various caches, CPU registers, read-only memory (ROM), and writable non-volatile memory, such as flash memory, hard drives, floppy disks, CDs, DVDs, magnetic storage devices, tape drives, and so forth. The memory can include program memory that stores programs and software, such as software for calculating a relaxation frequency from the measured tissue impedance.

### Method for Usage

The structure of the needle device permits the system 100 to be used in multiple applications of the tissue of interest, including but not limited to: measuring electrical properties of, performing a biopsy on, and/or delivering substances to the tissue of interest. The needle device can be placed in contact with the tissue of interest. Once the needle device is placed in contact with the tissue of interest, a biopsy can be performed wherein a sample of the tissue and/or fluid is extracted through the needle device 104. In another embodiment, the needle device 104 can be used to deliver substances in the opposite direction of an extraction/biopsy. For example, fluid can be passed through the needle device and introduced to the tissue, potentially for therapeutic purposes.

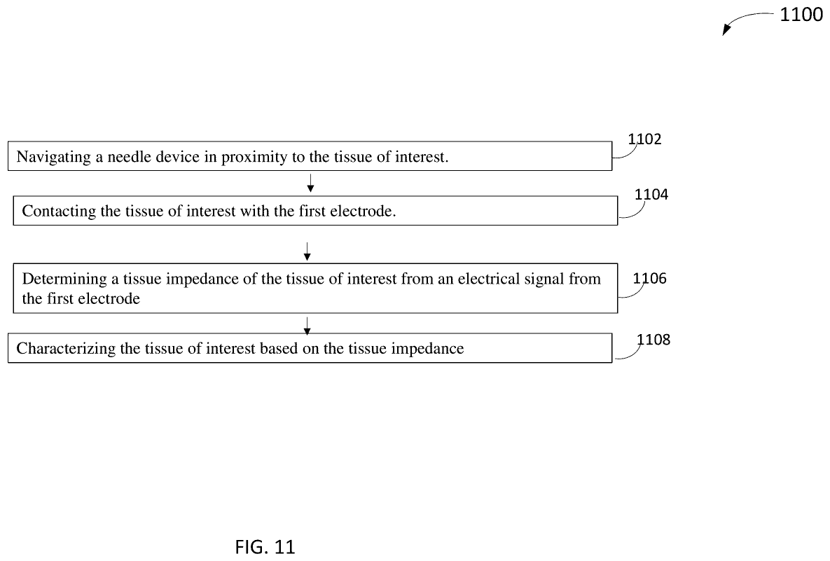

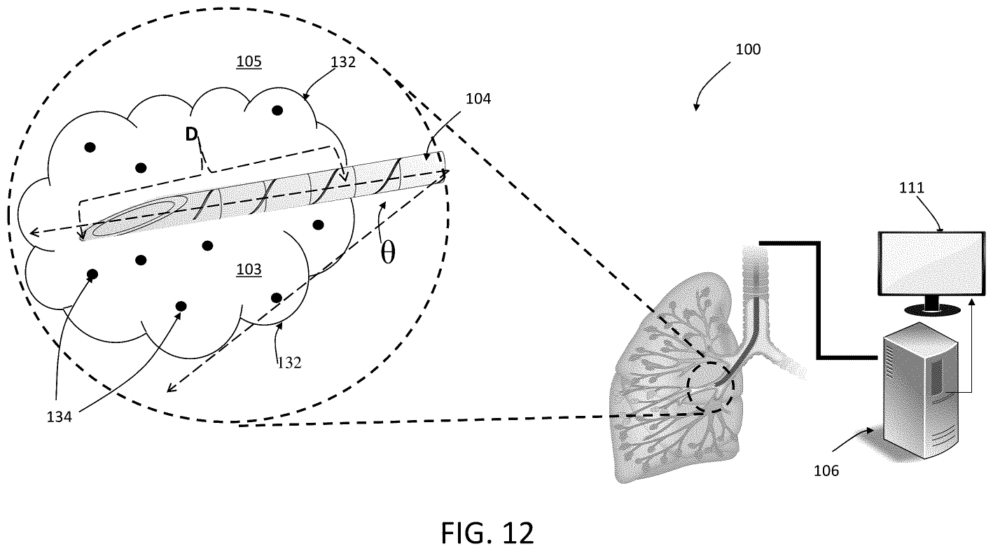

Referring back to the measurement of electrical properties of the tissue, as shown in FIG. 11, a method 1100 for determining the electrical impedance for a tissue of interest includes navigating a needle device in proximity to the tissue of interest (1102); contacting the tissue of interest with an electrode of the needle device (1104); determining a tissue impedance of the tissue of interest from an electrical signal from the electrode (1106); and characterizing the tissue of interest to be benign or malignant based on the tissue impedance (1108). Characterizing the tissue impedance can be based on an equivalent circuit embodied by the Cole function (1).

$$Z = \frac{{R}_{1} - {R}_{3}}{1 + {( j \frac{f}{{f}_{c}} )}^{α}} + {R}_{3} \qquad ( 1 )$$

where Z is the tissue impedance, R₁ and R₃ represent the low and high frequency limits of Z, tissue impedance; f is the measurement frequency; and f_{c} is the Cole relaxation frequency, both measured in hertz (Hz); j=√(−1); and a is a dimensionless number that is inversely related to the broadening in the frequency domain of Z. These quantities can be determined by the processor from the electrical signal received from the electrodes of the needle device.

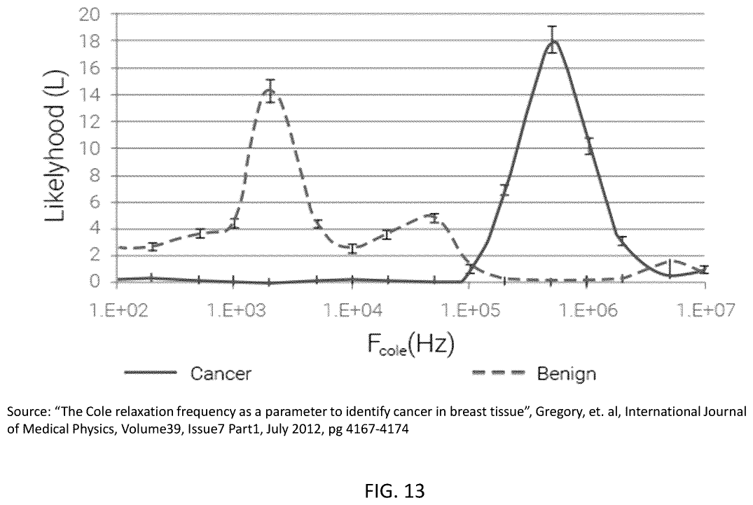

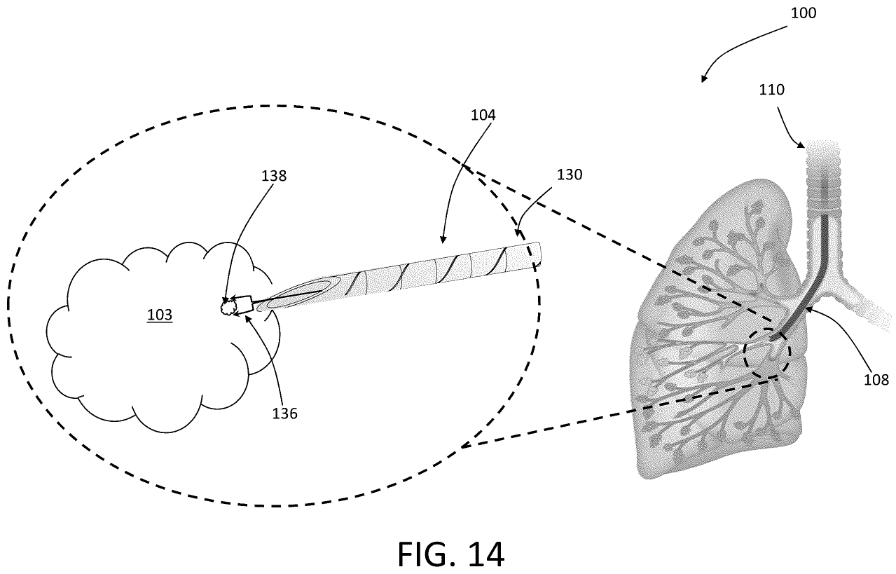

To characterize the tissue as malignant or benign, the processor can use the Cole relaxation frequency f_{c} derived from the Cole function using the measured tissue impedance (Z). The relaxation frequency can help optimize the characterization of the tissue because only the relaxation frequency is needed instead of a multiple variable analysis. In particular, without being bound by any particular theory, it is contemplated that as cells transform from benign to malignant, the cells are increasingly disorganized, based on partial wave optical scattering. This disorganization causes electrical polarization of cell contents to decrease, which then results in an increase in the relaxation frequency. In malignant tissue, the relaxation frequency can range between 1×10⁵ to 2×10⁶ Hz, as exhibited in FIG. 13. As depicted in FIG. 13, the relaxation frequency for benign tissue is orders of magnitude lower. The relaxation frequency can be normalized by dividing the range by 1×10⁵. The resulting scale can be a normalized range between 1 to 20. Thus, the higher the normalized range, the greater the likelihood the tissue of interest is malignant.

During usage, the needle device can be inserted through the working channel of an endoscope (e.g., manually controlled, or robotic-assisted bronchoscope, etc.) and into a tissue environment such as lung airways, see FIG. 1. In an example method in which the tissue of interest is in the lungs, the bronchoscope can be used to navigate the needle device. The bronchoscope can be introduced into the throat and trachea of a patient. The bronchoscope can then be maneuvered beyond the trachea and into the bronchial tree toward a target location near the tissue of interest. Once proximate the tissue of interest, the needle device can be extended from the bronchoscope to be placed in contact with the tissue of interest. The needle device 104 can then be used to take an initial impedance measurement of the tissue of interest. The measurements can be provided to the computing device 106 to characterize the tissue as benign or malignant. Additional steps can be taken to learn additional characteristics of the tissue of interest 103. For example, such additional characteristics can include size, volume, and more specific details about the location of the tissue of interest within the organ (e.g., lungs or kidney) while in vivo. Once a measurement is taken, the position of the needle device 104 can be adjusted. Referring to FIG. 12, when embodiment in tissue composition between the tissue of interest and surrounding tissue 105 is identified, the processor 106 can identify a boundary 132 between the tissue of interest and surrounding tissue 105. In a further embodiment, repetitively moving the needle device can identify additional boundary locations 132 between the surrounding tissue 105 and tissue of interest 103. In other configurations, the system 100 can perform impedance measurements on tissue invitro.

Furthermore, in some embodiments, the needle device 104 may be repositioned as desired to explore and characterize different portions of the tissue of interest. As depicted in FIG. 12, the needle device 104 can be inserted at variable depths (D) into the tissue of interest to further quantify the impedance values throughout the volume of the tissue of interest. In another embodiment, the angle of insertion (θ) can additionally or alternatively be adjusted to further quantify the impedance values for the tissue of interest. In further embodiments, understanding of the boundary 132 between the tissue of interest 103 and surrounding tissue 105 can be made by adjusting an insertion vector (e.g., depth (D) and/or angle (θ) of the needle device).

In another embodiment, the tissue of interest can involve kidney tissue. Similar to the process for performing a biopsy with lung tissue, the needle device can be introduced endoscopically (e.g., via a ureteroscope) through the urethra, bladder and/or ureter into the kidney, in order to perform a biopsy of kidney tissue. In a further aspect, the needle device can be introduced percutaneously to perform a biopsy of kidney tissue and/or other suitable tissue.

In some embodiments, the needle device 104 can yield additional measurement configurations when multiple electrodes are oriented along the longitudinal axis. In taking certain electrical property measurements, the electroconductive segments 120A-C can function as both a lead electrode and a reference electrode. For example, during a tissue impedance measurement, the needle device 104 can be inserted to a particular depth, the measured impedance variation across the electrodes at each segment can provide data for analysis by the computing device 106. The computing device 106 can determine whether the tissue of interest is malignant or benign tissue by the tissue impedance and resulting Cole relaxation frequency. Upon the determination of malignant tissue, additional information can be gained by inserting the needle device 104 deeper into the malignant tissue. During measurement, the computing device can also switch the role of lead electrode and reference electrode between the first electrode 120A, second electrode 120B and third electrode 120C to receive tissue impedance measurements at each electrode respectively. Additional embodiments of the electrode configuration can be achieved when the distance between electrodes along the member is non-uniform. For example, the distance between the first electrode 120A and second electrode 120B can be 5 mm, whereas the distance between the second electrode and the third electrode 120C can be 3 mm.

In another embodiment, the process of taking multiple impedance measurements by inserting the needle device to a certain depth can be used to define an arrangement of nodes 134. The arrangement of nodes 134 can provide additional understanding for varying tissue characteristics in the tissue of interest 103. The nodes can represent spatial regions in the tissue regions in the tissue of interest. The data associated with each node can give insight to the change in tissue properties across the volume of the tissue of interest. For example, a separate node 134 can be established to represent the measurement data at each electrode 120 of the member 107 while in contact with the tissue of interest. As more measurements are taken at various measurement angles and various measurement depths, more nodes can be generated.

The processor 106 can generate a nodal network that details the electrical property variances in the tissue of interest using the data from the nodes. The processor 106 can output the measurements and/or nodal network on the visual display 111 or transmit the data to another computing device. The number of nodes or data can also be increased based on the amount of measurements taken. Each measurement can yield additional data when the needle comprises multiple electrodes. For example, the needle device can comprise five (5) electroconductive segments wherein each segment is associated with a wire 128 (conductive path). Thus, in such an embodiment, the needle device can capture five measurements of electrical impedance in the tissue of interest.

In addition to measurement of electrical properties, the needle device can also be used to perform a biopsy. When being used for a biopsy, the needle device can be coupled with extraction fixtures. During use, the needle device can be placed in contact with the tissue of interest. Once the needle device 104 is placed in contact with the tissue of interest, extraction fixtures 136 can be passed through the lumen as depicted in FIG. 14. The extraction fixtures 136 can comprise hooks or tines that are configured to grab on to a sample 138 of the tissue of interest. The extraction fixtures 136 can then detach a sample of the tissue of interest. In yet a further aspect, the extraction fixture 136 can also comprise a stabilization fixture, such as a cryoprobe, that can be configured to stabilize the tissue of interest during extraction.

In another embodiment, an aspiration technique can be used to extract a sample. During an aspiration, the needle device suctions out a small sample of cells and/or fluid that is passed through the lumen of the needle device 104. In some embodiments, the needle device may be coupled to a vacuum source so to directly pass tissue and/or fluid through the needle device lumen itself. Additionally or alternatively, the aspiration process can involve a separate suction device passed through the lumen 122 to pull a sample from the tissue of interest 103. In yet a further aspect, the extraction method can include fixtures that include both a suction fixture and extraction fixture. In another embodiment, the needle device can also be used to introduce substances or fluids to the tissue of interest. Similar to the biopsy extraction or aspiration process, the needle device 104 can be placed in contact with the tissue of interest 103. Once in place, a fluid or substance can be passed through the needle device via the lumen to introduce the substance or fluid to the tissue of interest.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawings disclose any additional subject matter that is not within the scope of the claims below, the disclosures are not dedicated to the public and the right to file one or more applications to claim such additional disclosures is reserved.

What is claimed is:

1. A device comprising:
a member comprising a plurality of electroconductive segments arranged between a proximal end and a distal end of the member, wherein the plurality of electroconductive segments are electrically insulated from one another; and
a first conductive path, wherein a portion of the first conductive path is electrically coupled to a first electroconductive segment of the plurality of electroconductive segments such that the first electroconductive segment is configured as a first electrode.

2. The device of claim 1, further comprising a second conductive path, wherein a portion of the second conductive path is electrically coupled to a second electroconductive segment of the plurality of electroconductive segments such that the second electroconductive segment is configured as a second electrode.

3. The device of claim 1, wherein the member comprises at least one conduit defined in an exterior surface of the member, extending along at least one longitudinal portion of the member, wherein the first conductive path is oriented in the at least one conduit.

4. The device of claim 3, wherein the at least one conduit is equally circumferentially arranged around the member.

5. The device of claim 1, further comprising at least one insulated segment coupled to at least one of the plurality of electroconductive segments.

6. The device of claim 5, wherein the at least one insulated segment is interspersed between two electroconductive segments of the plurality of electroconductive segments.

7. The device of claim 1, further comprising at least one insulated segment coupled to at least one of the plurality of electroconductive segments, wherein the at least one insulated segment is interspersed between two electroconductive segments of the plurality of electroconductive segments.

8. The device of claim 1, wherein the member comprises a needle that defines a lumen.

9. The device of claim 8, further comprising a suction device configured to remove a substance through the lumen.

10. A method for characterizing a tissue of interest, the method comprising:
navigating a needle device in proximity to the tissue of interest, wherein the needle device comprises a plurality of electroconductive segments electrically insulated from one another, wherein a conductive path is defined on an exterior surface of at least one of the electroconductive segments, and wherein a portion of the conductive path is electrically coupled to a first electroconductive segment of the plurality of electroconductive segments such that the first electroconductive segment is configured as a first electrode;
contacting the tissue of interest with the first electrode;
determining a tissue impedance of the tissue of interest from an electrical signal from the first electrode; and
characterizing the tissue of interest based on the tissue impedance.

11. The method of claim 10, wherein characterizing the tissue of interest comprises determining the tissue of interest to be malignant or benign.

12. The method of claim 10, wherein determining the tissue impedance comprises identifying an electrical impedance value associated with the tissue of interest and transmitting the electrical signal associated with the electrical impedance value to a computing device.

13. The method of claim 10, wherein navigating the needle device in proximity to the tissue of interest further comprises placing the needle device in an endoscope and positioning the endoscope in proximity to the tissue of interest.

14. The method of claim 10, further comprising delivering a substance to the tissue of interest via the needle device.

15. The method of claim 10, further comprising extracting a fluid or a sample of the tissue of interest via the needle device.

16. The method of claim 10, the method further comprising repositioning the needle device relative to the tissue of interest.

17. The method of claim 16, wherein repositioning the needle device comprises changing an insertion depth of the needle device in the tissue of interest.

18. The method of claim 16, wherein repositioning the needle device comprises changing an insertion angle of the needle device in the tissue of interest.

19. The method of claim 10, wherein the needle device comprises a second conductive path electrically coupled to a second electroconductive segment of the plurality of electroconductive segments such that the second electroconductive segment is configured as a second electrode, wherein the method comprises contacting a first tissue region of the tissue of interest with the first electrode and contacting a second tissue region of the tissue of interest with the second electrode.

20. The method of claim 19, further comprising determining a first tissue impedance of the first tissue region and a second tissue impedance of the second tissue region based on electrical signals from the first electrode and the second electrode, respectively.
